SEQUENCE LISTING (73 sequences)

SEQ ID NO: 1 - protein, 14 aa, Homo sapiens
WESNGQPENNYKTT

SEQ ID NO: 2 - protein, 9 aa, Homo sapiens
SNGQPENNY

SEQ ID NO: 3 - protein, 14 aa, Artificial
  CD-2WNG loop epitope with border sequences
WESNGNELSDFKTT

SEQ ID NO: 4 - protein, 8 aa, Artificial
  CD-2WNG epitope
NGNELSDF

SEQ ID NO: 5 - protein, 14 aa, Artificial
  CD-4I2XA epitope with border sequences
WEIDGSERQNGKTT

SEQ ID NO: 6 - protein, 9 aa, Artificial
  CD-4I2XA epitope
IDGSERQNG

SEQ ID NO: 7 - protein, 10 aa, Artificial
  CD-TLK epitope with border sequences
WEDNPVYKTT

SEQ ID NO: 8 - protein, 5 aa, Artificial
  CD-TLK epitope
DNPVY

SEQ ID NO: 9 - protein, 14 aa, Artificial
  CD-CD2HV epitope with border sequences
WESNIAQPRNYKTT

SEQ ID NO: 10 - protein, 9 aa, Artificial
  CD-CD2HV epitope
SNIAQPRNY

SEQ ID NO: 11 - protein, 18 aa, Artificial
  CD-KRNE epitope with border sequences
WESNGQPEKRNENNYKTT

SEQ ID NO: 12 - protein, 13 aa, Artificial
  CD-KRNE epitope
SNGQPEKRNENNY

SEQ ID NO: 13 - protein, 18 aa, Artificial
  CD-LANE epitope with border sequences
WESNGQPELANENNYKTT

SEQ ID NO: 14 - protein, 13 aa, Artificial
  CD-LANE epitope
SNGQPELANENNY

SEQ ID NO: 15 - protein, 14 aa, Artificial
  CD-DRR epitope with border sequences
WESNGQPDRRYKTT

SEQ ID NO: 16 - protein, 9 aa, Artificial
  CD-DRR epitope
SNGQPDRRY

SEQ ID NO: 17 - protein, 14 aa, Artificial
  CD-DNF-derived epitope with border sequences
WESNGQPEDNFKTT

SEQ ID NO: 18 - protein, 9 aa, Artificial
  CD-DNF-derived epitope
SNGQPEDNF

SEQ ID NO: 19 - protein, 14 aa, Artificial
  CD-DQQ epitope with border sequences
WESNGQPDQQYKTT

SEQ ID NO: 20 - protein, 9 aa, Artificial
  CD-DQQ epitope
SNGQPDQQY

SEQ ID NO: 21 - protein, 14 aa, Artificial
  CD-OPN epitope with border sequences
WETWLNPDPSQKTT

SEQ ID NO: 22 - protein, 9 aa, Artificial
  CD-OPN epitope
TWLNPDPSQ

SEQ ID NO: 23 - protein, 13 aa, Artificial
  CD-Glu epitope with border sequences
WEYMPMENNYKTT

SEQ ID NO: 24 - protein, 8 aa, Artificial
  CD-Glu epitope
YMPMENNY

SEQ ID NO: 25 - protein, 14 aa, Artificial
  CD-Myc epitope with border sequences
WEQKLISEEDLKTT

SEQ ID NO: 26 - protein, 9 aa, Artificial
  CD-Myc epitope
QKLISEEDL

SEQ ID NO: 27 - protein, 12 aa, Artificial
  CD-FLAG epitope with border sequences
WEDYKDDDDKTT

SEQ ID NO: 28 - protein, 7 aa, Artificial
  CD-FLAG epitope
DYKDDDD

SEQ ID NO: 29 - protein, 15 aa, Artificial
  CD-HIS epitope with border sequences
WESNGHHHHHHYKTT

SEQ ID NO: 30 - protein, 10 aa, Artificial
  CD-HIS epitope
SNGHHHHHHY

SEQ ID NO: 31 - protein, 10 aa, Homo sapiens
RDELTKNQVS

SEQ ID NO: 32 - protein, 4 aa, Homo sapiens
LTKN

SEQ ID NO: 33 - protein, 10 aa, Artificial
  AB loop epitope with border sequences
KDELSKNQVS

SEQ ID NO: 34 - protein, 4 aa, Artificial
  AB loop sequence
LSKN

SEQ ID NO: 35 - protein, 10 aa, Artificial
  AB loop epitope and border sequences
QDELSKNQVS

SEQ ID NO: 36 - protein, 4 aa, Artificial
  AB loop sequence
LSKN

SEQ ID NO: 37 - protein, 10 aa, Artificial
  AB loop epitope with border sequences
RDEISKNQVS

SEQ ID NO: 38 - protein, 4 aa, Artificial
  AB loop sequence
ISKN

SEQ ID NO: 39 - protein, 10 aa, Artificial
  AB loop epitope
QDEISKNQVS

SEQ ID NO: 40 - protein, 10 aa, Artificial
  AB-ISRQ epitope with border sequences
RDEISRQQVS

SEQ ID NO: 41 - protein, 4 aa, Artificial
  AB-ISRQ epitope
ISRQ

SEQ ID NO: 42 - protein, 10 aa, Artificial
  AB-2WNG epitope with border sequences
RDETPQHQVS

SEQ ID NO: 43 - protein, 4 aa, Artificial
  AB-2WNG epitope
TPQH

SEQ ID NO: 44 - protein, 10 aa, Artificial
  AB-4I2XA epitope with border sequences
RDELTSGQVS

SEQ ID NO: 45 - protein, 4 aa, Artificial
  AB-4I2XA epitope
LTSG

SEQ ID NO: 46 - protein, 9 aa, Artificial
  AB-4I2XE epitope with border sequences
WETPEHKTT

SEQ ID NO: 47 - protein, 4 aa, Artificial
  AB-4I2XE epitope
TPEH

SEQ ID NO: 48 - protein, 10 aa, Homo sapiens
REEMTKNQVS

SEQ ID NO: 49 - protein, 4 aa, Homo sapiens
MTKN

SEQ ID NO: 50 - protein, 10 aa, Artificial
  AB loop epitope with border sequences
KEEMSKNQVS

SEQ ID NO: 51 - protein, 4 aa, Artificial
  AB loop epitope
MSKN

SEQ ID NO: 52 - protein, 10 aa, Artificial
  AB loop epitope with border sequences
QEEISKNQVS

SEQ ID NO: 53 - protein, 10 aa, Artificial
  AB epitope loop with border sequences
KEEISKNQVS

SEQ ID NO: 54 - protein, 10 aa, Artificial
  AB loop epitope with border sequences
QEEISKNQVS

SEQ ID NO: 55 - protein, 10 aa, Artificial
  AB loop epitope with border sequences
REEISRQQVS

SEQ ID NO: 56 - protein, 10 aa, Artificial
  AB loop epitope with border sequences
REETPQHQVS

SEQ ID NO: 57 - protein, 10 aa, Artificial
  AB loop epitope with border sequences
REELTSQQVS

SEQ ID NO: 58 - protein, 10 aa, Homo sapiens
DKSRWQQGNV

SEQ ID NO: 59 - protein, 6 aa, Homo sapiens
KSRWQQ

SEQ ID NO: 60 - protein, 10 aa, Artificial
  EF-2WNG epitope with border sequences
DLTRWDVGNV

SEQ ID NO: 61 - protein, 6 aa, Artificial
  EF-2WNG epitope
LTRWDV

SEQ ID NO: 62 - protein, 10 aa, Artificial
  EF-4I2XA epitope with border sequences
DKDRWERGNV

SEQ ID NO: 63 - protein, 6 aa, Artificial
  EF-4I2XE epitope
KDRWER

SEQ ID NO: 64 - protein, 11 aa, Artificial
  EF-4I2XE epitope with border sequences
WELDRWDVKTT

SEQ ID NO: 65 - protein, 6 aa, Artificial
  EF-4I2XE epitope
LDRWDV

SEQ ID NO: 66 - protein, 10 aa, Artificial
  EF-ND epitope with border sequences
DNDRWQQGNV

SEQ ID NO: 67 - protein, 6 aa, Artificial
  EF-ND epitope
NDRWQQ

SEQ ID NO: 68 - protein, 106 aa, Homo sapiens [sequence omitted]

SEQ ID NO: 69 - protein, 106 aa, Homo sapiens
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 70 - protein, 327 aa, Homo sapiens
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSTRHHHHHH

SEQ ID NO: 71 - protein, 328 aa, Homo sapiens
EEELQMIQPEKLLLVTVGKTATLHCTVTSLLPVGPVLWFRGVGPGRELIYNQKEGHFPRVTTVSDLTKRNNMDFSIRISSITPADVGTYYCVKFRKGSPENVEFKSGPGTEMALGAKPSAPVVLGPAARTTPEHTVSFTCESHGFSPRDITLKWFKNGNELSDFQTNVDPTGQSVAYSIRSTARVVLDPWDVRSQVICEVAHVTLQGDPLRGTANLSEAIRVPPTLEVTQQPMRVGNQVNVTCQVRKFYPQSLQLTWSENGNVCQRETASTLTENKDGTYNWTSWFLVNISDQRDDVVLTCQVKHDGQLAVSKRLALEVSTRHHHHHH

SEQ ID NO: 72 - protein, 214 aa, Homo sapiens
DIVITQSPKFMSTSVGDRVSITCKASQDVSTAVAWFQQKPGQSPKLLIYSASYRYTGVPDRFTGSGSGTDFTFTISSVQAEDLAVYYCQQHYSTPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 73 - protein, 325 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFLPPKPKDTLMISRTPEVTCVVVDVSHEDPEVKFNWYVDGVEVHNAKTKPREEQYNSTYRVVSVLHQDWLNGKEYKCKVSNKALPAPIEKTISKAKGQPRDPQVYTLPPSRDELTKNEVSLTCLVKGFYPSDIAVEWEYMPMENNYKTTPPVLDSDGSFFLYSKLTVDKSRWQQGNVFSCSVMHEALHNHYTQKSLSLPG

(19) United States
(12) Patent Application Publication
(10) Pub. No.: US 2021/0221914 A1
(43) Pub. Date: Jul. 22, 2021

(54) CH3 DOMAIN EPITOPE TAGS

(71) Applicant: IMMUNOME, INC., Exton, PA (US)

(72) Inventors: Matthew K. ROBINSON, Blue Bell, PA (US); Michael John MORIN, Salisbury, MA (US)

(73) Assignee: IMMUNOME, INC., Exton, PA (US)

(21) Appl. No.: 17/055,646

(22) PCT Filed: May 17, 2019
(86) PCT No.: PCT/US2019/032780

§ 371 Date: Nov. 16, 2020

Related U.S. Application Data
(60) Provisional application No. 62/672,738, filed on May 17, 2018.

(51) Int. Cl. C07K 16/42

(52) U.S. Cl. CPC C07K 16/4241; C07K 2317/526; C07K 2318/10; C07K 2317/24

(57) ABSTRACT

This invention relates to the incorporation of one, or more, heterologous antibody epitopes into the AB, EF, or CD structural loops of the constant heavy domain 3 (“CH3 domain”) of an engineered antibody or Fc-linked therapeutic agent. The heterologous epitopes serve as “epitope tags” that are specifically detectable by epitope tag-specific detector antibodies, irrespective of the tagged agent's target specificity. Therefore, the epitope tags are useful for the rapid detection of any tagged antibody or Fc-linked agent in biological samples, including samples, which also contain endogenous antibodies.

### FIELD OF THE INVENTION

The field of this invention relates to the use of heterogenous antibody epitopes to facilitate detection of antibody-based biologics in a biological sample.

### BACKGROUND

Antibody-based biologics, such as therapeutic antibodies and Fc fusion proteins, are commonly developed on a human immunoglobulin G (IgG) scaffold to minimize undesirable recipient-mediated immune responses to a biologic following its administration. However, because humans naturally produce systemically circulating IgG, the IgG scaffold context of the administered biologic makes detection of the biologic within patient samples difficult due to the background presence of the endogenous human IgG. Having the ability to detect biologics in patient samples is important, because assays for tracking serum levels and pharmacokinetic (“PK”) behavior of biologics is routinely useful for the optimization of dosing of biologics.

Practitioners generally rely on anti-idiotypic monoclonal antibodies to detect the unique Fab epitopes idiotypes of antibody-based biologics to detect them against an endogenous IgG background. However, the development of each anti-idiotypic monoclonal antibody is time- and resource-intensive because each individual antibody-based biologic requires its own detection antibody.

Alternatively, the need to generate anti-idiotypic antibodies can be eliminated by incorporating one or more non-naturally occurring epitopes into the AB, EF, or CD loops of a CH3 scaffold derived from a human IgG Fc region, which, in turn, is incorporated into an antibody-based biologic.

### SUMMARY OF THE INVENTION

This invention relates to the inclusion of heterogenous antibody epitopes into antibody-based biologics to facilitate detection of the biologic against a background of endogenous antibodies, typically in the context of a patient sample. More specifically, heterogenous antibody epitopes are incorporated into one or more of the AB, EF, or CD structural loops of an IgG1-derived CH3 scaffold, which, in turn, is incorporated into an antibody-based biologic. In essence, the heterogenous epitope, or epitopes, of a CH3 scaffold according to the invention serves as an “epitope tag” to enable the rapid identification of proteins or complexes of proteins that comprise an epitope-tagged CH3 domain.

Moreover, the same epitope can be incorporated into a practically limitless number of different antibody-based biologics. Therefore, a rapid biologic detection system according to the invention can be generalized for use with different biologics, whereas conventional methods for detecting a biologic in a sample rely on the individualized use of a different anti-idiotype antibody for every different biologic.

### BRIEF DESCRIPTION OF THE FIGURES

FIG. 1 depicts a crystal structure of a complex between neonatal Fc Receptor (“FcRn”), human serum albumin (HSA), and an Fc region, as represented by PDB 4N0U, and visualized using PyMOL molecular modeling software. Chain A of the crystal structure is IgG receptor FcRn large subunit p51 (depicted in dark gray ribbon). Chain B of the crystal structure is β2 microglobulin subunit (depicted in light gray ribbon). Chain D (HSA) is not depicted. Chain E is one subunit of the IgG1 Fc region homodimer (light gray cartoon with highlighted regions in black). Residues highlighted in black sticks represent the CD and AB/EF loops. Fc residues depicted in black cartoon, without sticks, are within 5 Å of the Fc:FcRn interface, and overlap with residues predicted to be important for the dimerization.

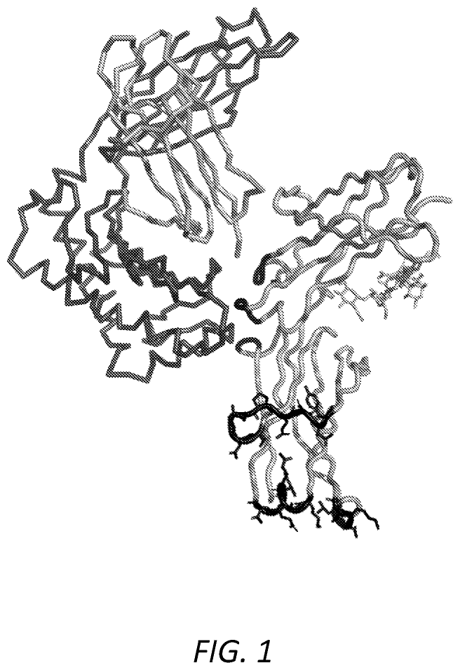

FIG. 2A shows a multi-sequence alignment of amino acid sequences of various human Ig-fold domain proteins, whose crystal structures are available in the Research Collaboratory for Structural Bioinformatics Protein Data Base (“PDB”), with amino acids 104-108 of PDB 4W12:A, corresponding to a region of the human IgG1 CH3 domain sequence. The alignment was performed using the sequence alignment software program, MAFFT.

FIG. 2B shows a multi-sequence alignment of amino acid sequences of various human Ig-fold domain proteins, whose crystal structures are available in the Research Collaboratory for Structural Bioinformatics Protein Data Base (“PDB”), with amino acids 109-202 of PDB 4W12:A, corresponding to a region of the human IgG1 CH3 domain sequence. The alignment was performed using the sequence alignment software program, MAFFT.

FIG. 2C shows a multi-sequence alignment of amino acid sequences of various human Ig-fold domain proteins, whose crystal structures are available in the Research Collaboratory for Structural Bioinformatics Protein Data Base (“PDB”), with amino acids 203-208 of PDB 4W12:A, corresponding to a region of the human IgG1 CH3 domain sequence. The alignment was performed using the sequence alignment software program, MAFFT.

FIG. 3 shows the alignment of amino acid sequences representing a select group of Ig-fold domain proteins from those depicted in FIG. 2 with a human a human IgG1 CH3 domain sequence derived from PDB 4W12.

FIG. 4 depicts a crystal structure of a wild-type human Fc region derived from the sequence of PDB 4W12, as visualized in gray cartoon using PyMOL molecular modeling software. Carbohydates are depicted in gray colored sticks. The AB, CD, and EF loops are depicted in black. Surface exposed side-chains within the AB and EF loops are represented with sticks. Surface exposure of side chains is predictive of their potential for contact with an antibody.

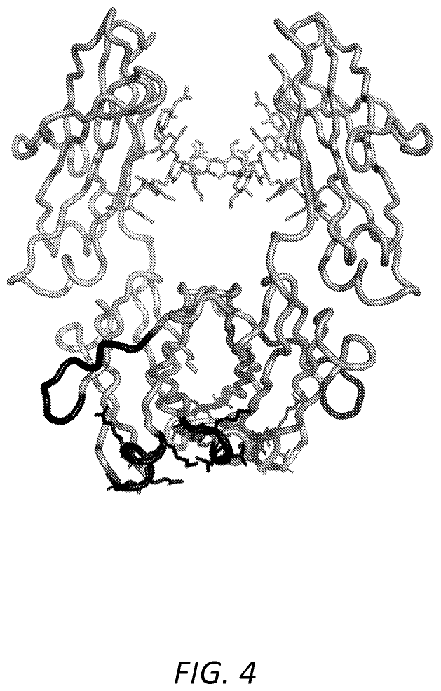

FIG. 5 depicts a crystal structure of a wild-type human Fc region derived from the sequence of PDB 4W12, as visualized in gray cartoon using PyMOL molecular modeling software. Carbohydrates are depicted in gray colored sticks. The AB, CD, and EF loops are depicted in black. Surface exposed side-chains within the CD loop are represented with sticks. Surface exposure of side chains is predictive of their potential for contact with an antibody.

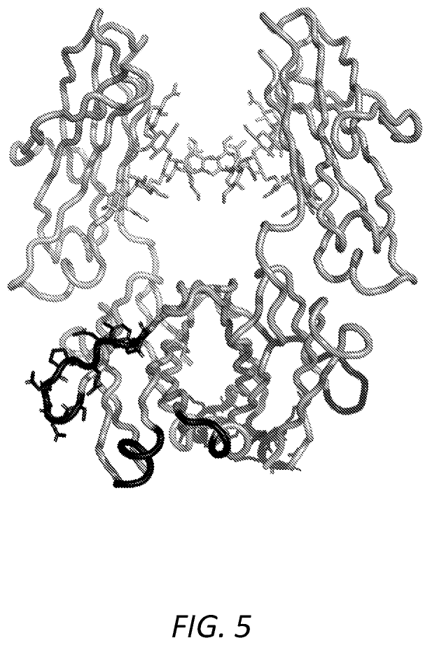

FIG. 6 depicts the surface of an AB-EF loop region of a human Fc region. Surface residues in the AB-EF loops are depicted as spheres. The PDB structure 4W12 was visualized using PyMOL software package.

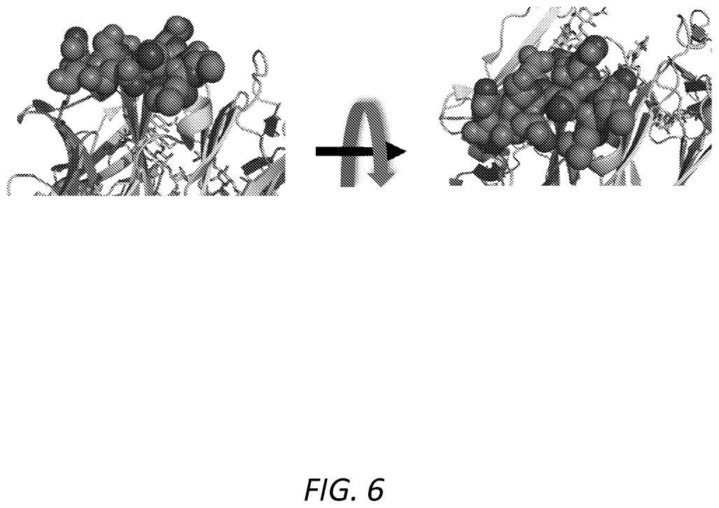

FIG. 7 shows Modeled Surface Residues in the AB and EF loops. PyMOL visualization of PDB #4W12 that was mutated to incorporate SEQ ID NO.38 and SEQ ID NO.67, in the AB and EF loops respectively, to generate the epitope tag.

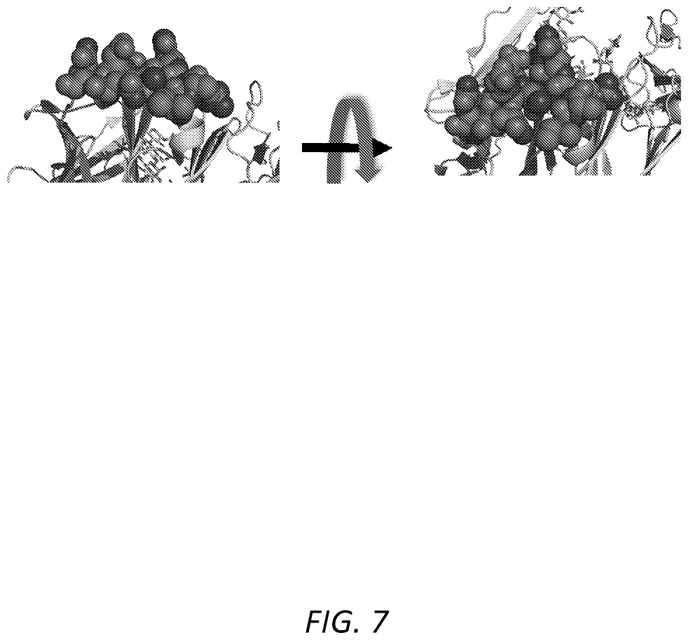

FIG. 8 is scanned image of a Western blot assessing the ability of anti-Glu antibody to detect human antibodies with either a wild-type or tagged (CD-Glu or CD-412X) Fc domain.

FIG. 9. depicts an ELISA-based detection of CD-GLU in the presence or absence of various ratios (microgram/mL:microgram/mL) of CD-WT antibody.

FIG. 10 depicts data from a competitive binding FRET experiment evaluating the ability of antibodies containing a series of different tags, to competitively Inhibit binding of antibody with a wild-type (m1,17) Fc to FcRn

FIG. 11 depicts data from a competitive FRET assay evaluating the ability of antibodies containing a series of different tags, to competitively inhibit binding of antibody with a wild-type (m1,17) Fc to CD16a.

FIG. 12 SPR-based affinity measurements of m1,17 and CD-GLU to series of Fc receptors.

FIG. 13 plots on-rates (ka, 1/Ms) versus off-rates (kd, 1/s) of CD-WT (dark gray) and CD-GLU (light gray) for binding to FcγRI. Data point to a small (1.2-fold) increase in the off-rate for CD-GLU relative to CD-WT.

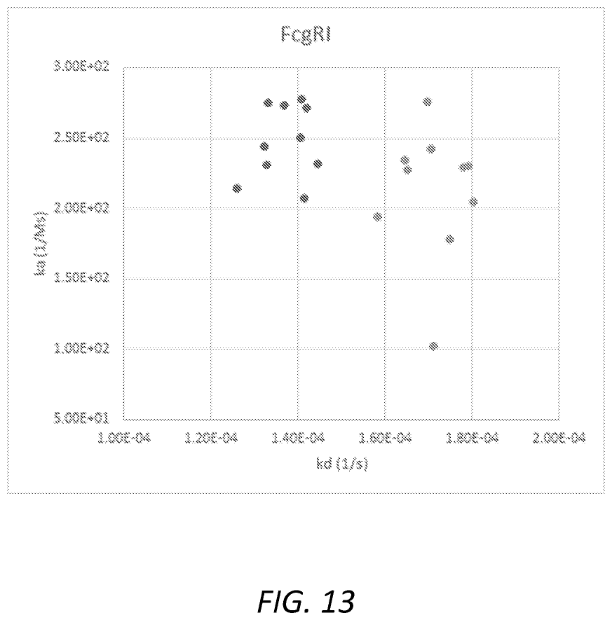

### DETAILED DESCRIPTION

The invention is directed to compositions and methods related to the incorporation of one or more heterologous antibody epitopes into a constant heavy domain 3 (“CH3 domain”) of an immunoglobulin Fc structure, or CH3-containing fragment thereof. For example, the invention can incorporate a heterologus epitope into a CH3 domain of a human IgG antibody. Such a CH3 domain-incorporated epitope according to the invention can serve as an “epitope tag” to allow the rapid identification of proteins or complexes of proteins that comprise an epitope-tagged CH3 domain. In general, the amino acid sequence of a CH3 domain epitope tag according to the invention is also derived or modified from a CH3 domain, and retains the basic tertiary structure of a CH3 domain, and thus, is also referred to herein as a “CH3 scaffold”. In other words, a CH3 domain epitope tag exists within the context of a “CH3 scaffold”. A CH3 scaffold derived from a human IgG1 molecule is the preferred structural context for a CH3 scaffold according to the invention, though a CH3 scaffold derived from any CH3 domain-possessing immunoglobulin molecule, such as human IgG2, IgG3, or IgG4. Likewise, a CH3 scaffold according to the invention can also be derived or modified from a structural domain of a non-immunoglobulin protein, which possesses a tertiary structure that is, at least, in part, conserved with respect to an IgG CH3 domain.

Indeed, a CH3 scaffold, according to the invention, substantially retains the structural characteristics of a naturally-occurring CH3 domain, known as an immunoglobulin fold (Ig-fold), including the packing of two beta sheets of a naturally occurring CH3 domain, i.e., the 3-stranded beta sheet containing antiparallel beta strands C, F, and G, packed against the 4-stranded beta sheet containing beta strands A, B, D, and E, arranged in antiparallel orientation. Amino acid residues involved in maintaining the packing of the beta sheets are known in the art, including the residues that form hydrogen bonding, hydrophobic interactions, and the disulfide bond. In specific embodiments, the residues critical to maintaining the Ig-fold are not modified. In certain embodiments, the framework residues are substantially not modified; for example, not more than 15%, or 10% or 5% of the framework residues are modified in an engineered CH3 scaffold as compared to a wild type CH3 domain. Modifications at or near the loop connecting either two beta strands of a beta sheet (e.g. AB loop) or strands of two different sheets (e.g. CD or EF loops) of a native CH3 may be more tolerable (i.e., less likely to disrupt the structure or conformation of a native CH3) as compared to modifications to other regions. CH3 scaffolds, in the context of an immunoglobulin heavy chain (“IHC”), retain the FcRn binding structure of a wild type CH3 molecule. For example, the residues which are believed to be critical to the FcRn binding function.

Proteins, which have been engineered to contain an epitope-tagged CH3 scaffold according to the invention are, in general, therapeutic antibodies (An antibody suitable for administration to subjects for treatment or prevention of a disease or disorder) and Fc-linked therapeutic products such as Fc-fusion proteins and Fc-linked drugs or therapeutic agents, which can be referred to, collectively, as Fc-biologics.

Antibodies according to the invention are preferably monoclonal antibodies. A monoclonal antibody is generally understood to have been produced by a single clonal B-lymphocyte population, a clonal hybridoma cell population, or a clonal population of cells into which the genes of a single antibody, or portions thereof, have been transfected. Monoclonal antibodies are produced by methods known to those of skill in the art, for instance by making hybrid antibody-forming cells from a fusion of myeloma cells with immune lymphocyte cells.

Monoclonal antibodies according to the invention also include humanized monoclonal antibodies. More specifically, a “human” antibody, also called a “fully human” antibody, according to the invention, is an antibody that includes human framework regions and CDRs from a human immunoglobulin. For example, the framework and the CDRs of an antibody are from the same originating human heavy chain, or human light chain amino acid sequence, or both. Alternatively, the framework regions may originate from one human antibody, and be engineered to include CDRs from a different human antibody.

The epitope-tagged antibodies according to the invention may be monospecific, bispecific, trispecific or of greater multi specificity. Multispecific antibodies may be specific for different epitopes of a polypeptide or may be specific for heterologous epitopes, such as a heterologous polypeptide or solid support material. See, e.g., PCT publications WO 93/17715; WO 92/08802; WO 91/00360; WO 92/05793; U.S. Pat. Nos. 4,474,893; 4,714,681; 4,925,648; 5,573,920; 5,601,819.

Examples of therapeutic antibodies which can be engineered to include a CH3 scaffold according to the invention include, but are not limited to: Chimeric mouse/human IgG1 targeting CD20 (e.g., rituximab); Humanized IgG1 targeting HER2 (e.g., trastuzumab); Humanized IgG1 targeting CD52 on B and T lymphocytes (e.g., alemtuzumab); human IgG2 targeting RANKL (e.g., denosumab); Humanized IgG4 tageting alpha-4 integrin (e.g., natalizumab); human IgG2 targeting EGFR, ErbB-1 and HER1 (e.g., panitumumab); Humanized IgG2/4k targeting complement protein C5 (e.g., eculizumab); Chimeric mouse/human IgG1 targeting EGFR, ErbB-1 and HER1 (e.g., cetuximab); Humanized IgG1 targeting VEGF (e.g., bevacizumab); human IgG1 targeting TNF-alpha (e.g. adalimumab); and Chimeric mouse/human IgG1 targeting TNF-α (e.g., infliximab).

As with epitope-tagged antibodies of the invention, the Fc region of a Fc-fusion protein according to the invention is also preferably derived from a human Immunoglobulin G (“IgG”) class framework, and more particularly an IgG1 subclass. An Fc fusion protein may be a monomeric protein or a multimeric protein, such as a dimeric or tetrameric protein, which may be formed by multimerisation via its Fc region. The Fc region provides the PK behavior of an Fc fusion protein.

In general, Fc-fusion proteins are bioengineered polypeptides that join the crystallizable fragment (Fc) region of an antibody with another biologically active protein domain or peptide to generate a molecule with unique structure—function properties and therapeutic potential. Fc-fusion proteins, in which the Fc region is fused to an extracellular domain of a native form of a receptor, can act as traps for ligands. Examples of Fc fusion proteins, which can be engineered to include a CH3 scaffold according to the invention include, but are not limited to: CTLA-4 fused to the Fc region of human IgG1 (e.g., belatacept); VEGFR1/VEGFR2 fused to the Fc region of human IgG1 (e.g., aflibercept); IL-1R fused to the Fc region of human IgG1 (e.g., rilonacept); Thrombopoietin-binding peptide fused to the Fc region of human IgG1 (e.g., romiplostim); Mutated CTLA-4 fused to the Fc region of human IgG1 (e.g., abatacept); LFA-3 fused to the Fc region of human IgG1 (e.g., alefacept); and TNFR fused to the Fc region of human IgG1 (e.g., etanercept).

Antibodies according to the invention, which are used to detect epitope tagged CH3 scaffolds, and proteins that comprise epitope tagged CH3 scaffolds, are generally referred to herein as “detector antibodies”. As used herein, the term “specifically binds” or “specific binding” refers to a binding reaction which is determinative of the cognate ligand of interest in a heterogeneous population of molecules. Thus, under designated conditions (e.g. immunoassay conditions), a detector antibody according to the invention binds to its particular target, such as a CH3 epitope tag according to the invention, and does not bind in a significant amount to other molecules present in a sample, such as endogenous antibodies in a patient sample. Specific binding means that binding is selective in terms of affinity for its target, and is usually achieved if the binding constant or binding dynamics is at least 10 fold different, preferably the difference is at least 100 fold, and more preferred a least 1000 fold.

The term “epitope” refers to a structure, typically formed by a sequence of amino acids, capable of being specifically bound by an antibody structure, including naturally-occurring and monclonal antibodies, as well as fragments of such molecules. In other words, an epitope can be a molecular structure which may completely make up a specific binding partner or be part of a specific binding partner to the binding domain of an antibody, or fragment thereof. The epitope tag of a CH3 scaffold according to the invention will usually include at least 3 amino acids, preferably 5 to 15 amino acids, or 10 to 20 amino acids, and may include one or more amino acids that border the modified AB, EF, or CD loop sequences of an epitope tag. Furthermore, while epitopes are generally linear, an epitope according to the invention, can also be conformational; for example, an epitope formed by the bringing together of noncontiguous sequences by folding of a polypeptide to form the tertiary structure of an epitopes.

Epitope tags, according to the invention, are characterized by at least one modification of the wild-type amino acid sequence of AB, EF, or CD loops of a CH3 scaffold, or any combination thereof that results in the formation of an epitope that is not recognized by an endogenous antibody produced by an individual in response to a therapeutic antibody, or Fc-fusion protein comprising an epitope-tagged CH3 scaffold. Various strategies may be used in the design of epitope tags. For example, sequence modifications within the AB, CD, and EF loops can be based on the substitution of AB, CD, or EF loop wild-type sequences with sequences derived from corresponding structural regions of other structurally-related Ig-fold proteins. Thus, candidate epitope sequences can be identified by performing a multi-sequence alignment of the primary amino acid sequences of various Ig-fold proteins against the sequence of the CH3 domain of an IHC. For example, primary amino acid sequences of Ig-fold protein crystal forms catalogued in the Research Collaboratory for Structural Bioinformatics Protein Data Bank (“PDB”) can be aligned against the primary sequence of an IHC CH3 domain.

An alignment analysis can consider general classes of sequence position conservation, including spacing within IgG folds, amino acid charge, isoelectric point (pI), polarity, and conservation of three-dimensional (3D) structure, as informed by crystal structure comparisons. A sequence alignment can also emphasize absolute identity at a conserved sequence predicting the amino acid is essential for maintaining the tertiary structure of the CH3 domain, generally, or an AB, EF, or CD loop structure, specifically. Such amino acids may also be called “anchor residues”. For example, the Val-Ser dipeptide sequence, C-terminal to the AB loop, and the Trp located two residues N-terminal to the CD loop are anchor residues. If a sequence alignment reveals the absence of an anchor residue in an otherwise conserved candidate Ig fold-derived epitope, the wild type sequence of the donor Ig fold protein can be modified by a substitution of the amino acid at the position in the donor sequence with the anchor residue corresponding to its position in the wild type CH3 sequence.

Examples of Ig fold proteins with Ig fold domains, from which epitope tag amino acid sequences according to the invention, may be derived are signal regulatory protein alpha (SIRPα) and SIRP gamma (SIRPγ). Crystal forms of SIRPα and SIRPγ are identified as 2WNG and 412X:E in the PDB. For example, according to the invention, the amino acids at the positions in the AB loop of an IHC, which correspond with a wild type sequence, such as, LTKN (SEQ ID NO. 32), can be substituted with the SIRPα and SIRPγ derived sequences TPQH (SEQ ID NO. 43) and TPEH (SEQ ID NO.47), respectively. It can also be substituted with the light chain constant domain (“CL”)-derived sequence LTSG (SEQ ID NO. 45), respectively.

Similarly, the amino acids at the positions in the EF loop of an IHC, which correspond with a wild type sequence, such as, KSRWQQ (SEQ ID NO. 59), can be substituted with SIRPα-, SIRPγ-, and CL-derived sequences, such as LTRWDV (SEQ ID NO. 61), LDRWDV (SEQ ID 65), and KDRWER (SEQ ID NO. 63), respectively. More particularly, SEQ ID NOS. 61, 65, and 63 correspond with positions: 186-191; 187-192; and 183-188, of the SIRPα, SIRPγ, and CL sequences described by SEQ ID NOS. 70, 71 and 72, respectively. The sequence, “RW”, in SEQ ID NOS. 61, 65, and 63, replaces wild type sequences, “RE”, “PW”, and “EY” at corresponding sequence positions in SIRPα, SIRPγ, and CL, respectively. More specifically, the “RW” sequence recapitulates the presence of RW at the corresponding sequence position in the EF loop of a wild type CH3 domain. Therefore “RW” serves as an anchor sequence to preserve the overall structure of a CH3 scaffold.

SIRPγ and SIRPα also contain regions that correspond with amino acids at the positions in the CD loop of an IHC, which correspond with a wild type sequence, such as, SNGQPENNY (SEQ ID NO. 2). Indeed, a CH3 wild type sequence can be substituted with the SIRPγ and SIRPα derived sequence NGNELSDF (SEQ ID NO. 4). The wild type sequence of the CH3 CD loop can be substituted with CL-derived sequence IDGSERQNG (SEQ ID NO. 6). SEQ ID NOs. 6 and 4 correspond with positions 150-158 and 156-163 of the CL and SIRPα sequences described by SEQ ID Nos. 72 and 70, respectively.

An additional strategy for designing epitope tags involves selecting sequence modifications of AB, EF, and CD loops that alter the wild type sequence, while preserving the overall 3D structure of the loops, including the avoidance of modifications that would create undesirable steric effects. Accordingly, a CH3 scaffold, according to the invention, may contain amino acid substitutions, deletions, or insertions to AB, EF, or CD loop sequences, in which properties of the wild type loop sequence amino acids, such as charge, pI, and polarity, may be preserved to maintain a natural framework for the epitope tag, but one in which the absolute sequence identity of solvent exposed amino acids (i.e., surface accessible to an epitope-specific antibody), is altered to favor specific binding by a detector antibody.

Various strategies for considering 3D structural and steric relationships when designing novel epitope tags for a CH3 scaffold are known in the art. For example, a molecular visualization system software, such as PyMOL, can be used to model candidate AB, EF, and CD loop epitope tag sequences in the context of an antibody, like, but not limited to the human IgG1 Fc regions derived from crystal structures of PDB 4W12 or 4N0U.

A non-limiting example of an AB loop epitope tag, in accordance with the invention, contains a substitution of a wild type sequence, such as LTKN (SEQ ID NO. 32) with ISRQ (SEQ ID NO. 41). Whereas, a non-limiting example of an EF loop epitope tag, in accordance with the invention, substitutes a wild type sequence, such as KSRWQQ (SEQ ID NO. 59) with NDRWQQ (SEQ ID NO. 67). Non-limiting examples of CD epitope tag sequences, according to the invention include the following sequences, which generally replace a IgG1 wild type sequence, such as, SNGQPENNY (SEQ ID NO. 2), with: DNPVY (SEQ ID NO. 8); SNIAQPRNY (SEQ ID NO. 10); SNGQPEKRNENNY (SEQ ID NO. 12); SNGQPELANENNY (SEQ ID NO. 14); SNGQPDRRY (SEQ ID NO. 16); SNGQPDNF (SEQ ID NO. 18); or SNGQPDQQY (SEQ ID NO. 20).

As stated above, epitope tags, according to the invention, are characterized by at least one modification of the wild-type (WT) amino acid sequence of AB, EF, or CD loops of the CH3 domain, or any combination thereof. Accordingly, an IHC, or portion thereof, possessing a CH3 domain, according to the invention, can have a single epitope tag located within only its AB loop, its EF loop, or its CD loop. Alternatively, an IHC, or portion thereof, possessing a CH3 domain, according to the invention, can have only two epitope tags, with one epitope tag located within its AB loop, and the other in its EF loop. Likewise, an IHC, or portion thereof, possessing a CH3 domain, according to the invention, can have only two epitope tags, with one epitope tag located within its AB loop, and the other in its CD loop. It also follows that an IHC, or portion thereof, possessing a CH3 domain, according to the invention, can have only two epitope tags, with one epitope tag located within its EF loop, and the other in its CD loop. For uses requiring an IHC, or portion thereof, possessing a CH3 domain, to have three epitope tags according to the invention, the IHC, or portion thereof will contain three epitope tags located at its AB, EF, and CD loops, respectively.

As stated above, antibody-based biologics based on, or in the context of, the human IgG1 subclass are preferred according to the invention. More particularly, the Fc region of an epitope-tagged, antibody-based biologic can be derived from various IgG1 allotypes (Jefferis & Lefranc). For example, the Fc region may be derived from an IgG1 antibody having the primary amino acid sequence of either a G1m1, or a nG1m1, allotype. As the G1m1 and nG1m1 allotypes are naturally distinguished by differences in their amino acid sequences within the AB loop, the design of an AB loop epitope tag can preserve those sequence differences by maintaining sequence identity, at those positions. More specifically, the wild-type G1m1 allotype includes amino acid sequence, RDELTKNQVS, and the corresponding nG1m1 sequence is REEMTKNQVS. The amino acids highlighted in bold in the foregoing sequences are the specific determinants of the allotype. Thus, the presence of the highlighted E and M residues in the AB loop of a nG1m1-derived Fc region would prevent a G1m1-specific antibody from binding to the Fc region. Similarly, the presence of the highlighted D and L residues in the AB loop of a G1m1-derived Fc region prevent a nG1m1-specific antibody from binding to the Fc region.

Allotypes may also exist within CH1 domain of the IHC of an IgG1 antibody. The IHC of an epitope-tagged, antibody-based biologic can be derived from various IgG1 allotypes. For example, the IHC may be derived from an IgG1 antibody having the primary amino acid sequence of either a G1m3 (IMGT R120; www.imgt.org), or a G1m17 (IMGT K120), allotype. More particularly, the IHC of an epitope-tagged antibody, antibody-based biologic can be derived from a combination of allotypes. More specifically, the IHC could be the G1m17,1 (“m1,17”) allotype that incorporates the combination of G1m1 and G1m17 allotypes.

Further to the foregoing consideration of allotype sequences in the design of epitope sequences, an epitope tag according to the invention can, but is not required to, include wild-type amino acid sequences that border the portion of the epitope tag that was particularly designed to function as an epitope, using, for example, the strategies discussed above. For example, an AB loop epitope tag sequence, which is bordered by (amino/carboxy) border sequences associated with a wild type G1m1 allotype of IgG1. Similarly, an AB loop epitope tag sequence, which is bordered by (amino/carboxy) border sequences associated with a wild type nG1m1 allotype of IgG1.

An EF loop epitope tag sequence, which is bordered on its amino- and carboxy-terminal sides by IgG1 wild type sequences (D) and (GQV), respectively, can also include a portion, or all, of the foregoing wild type sequences. Finally, a CD loop epitope tag sequence, which is bordered on its amino- and carboxy-terminal sides by IgG1 wild type sequences (WE) and (KTT), respectively, can also include a portion, or all, of the foregoing wild type sequences.

The invention also includes polynucleotides which encode a CH3 scaffold according to the invention. For example a polynucleotide according to the invention can encode a single CH3 scaffold polypeptide, or a polypeptide or fraction thereof containing a CH3 scaffold according to the invention, such as an IHC, an antibody fragment, or component of an Fc fusion protein.

Polynucleotides encoding the molecules of the invention may be obtained by any method known in the art. Indeed, well-known molecular biology methods can be employed to design and produce a polynucleotide that encodes a CH3 scaffold having AB, EF, and CD structural loop regions, in which at least one of the structural loop regions comprises an antibody epitope amino acid sequence. As stated above, an antibody epitope amino acid sequence according to the invention, contains at least one sequence modification of at least one of a CH3 scaffold's AB, EF, or CD structural loop regions. Therefore, the nucleotide sequence of a polynucleotide encoding a CH3 scaffold according to the invention can include sequence modifications that result in the expression of a CH3 scaffold with at least one amino acid substitution, deletion or insertion within at least one of its AB, EF, or CD loops relative to a wild-type CH3 domain sequence. In that regard, a polynucleotide according to the invention can contain a modified nucleotide sequence, in which the nucleotide sequence is modified to express a CH3 scaffold in which one or more AB, EF, or CD loops contain an amino acid sequence derived from a structurally related Ig fold protein. For example, a polynucleotide according to the invention can contain the nucleotide sequence encoding a CH3 scaffold, in which one or more of its AB, EF, or CD loops contain an amino acid sequence derived from the Ig fold proteins, SIRPα, SIRPγ, or another immunoglobulin chain, such as a constant light chain.

A polynucleotide according to the invention can be incorporated into a protein expression vector, which, in turn, can be transfected into a protein expression system host cell to drive the expression of a CH3 scaffold or CH3 scaffold-containing protein, such as an IHC, an antibody fragment, or component of an Fc fusion protein.

The invention also provides for methods for screening and identifying molecules, including antibodies and antigen-binding fragments, that specifically bind an engineered epitope of a CH3 scaffold according to the invention. The molecule that binds the epitope may, for example, be a monoclonal antibody or antigen-binding fragment thereof. Also provided are methods or processes for producing antibodies and antigen-binding fragments thereof that react with an epitope-tagged CH3 scaffold according to the invention.

While the invention does not place limitations on the availability of methods for identifying epitope-binding molecules, an example of such methods includes: (i) screening a biological sample or a peptide library using an epitope-tagged CH3 scaffold according to the invention as a probe; (ii) isolating a molecule that specifically binds the probe; and (iii) identifying the molecule. Therefore, an antibody or antibody fragment which specifically binds an engineered epitope of a CH3 scaffold according to the invention, can be used to detect an antibody having an epitope-tagged CH3 scaffold in a sample containing an excess of untagged antibodies or antibody fragments. For example, an antibody which specifically binds an engineered epitope of a CH3 scaffold according to the invention can specifically distinguish and detect engineered epitope-tagged (“tagged”) antibodies in a solution which also contains untagged antibodies at ratios of tagged: untagged of at least 1:250000, 1:100000, 1:10000, 1:1000, 1:100, or any ratio therein.

### EXAMPLES

The following Examples describe the design and analysis processes of the amino acid sequence within the AB, CD, and EF loops of the CH3 domain of human IgG to create epitope tags to allow easy detection of specialized antibodies in a sample using antibody cognates of the tags. Four general strategies were employed for designing epitope tag sequences for AB, EF, and CD loops: i) the substitution of wild-type sequences with sequences derived from regions of other Ig-fold proteins that share sequence or structural similarities with the CH3 loop structures, or both; ii) the use of molecular modelling software to identify sterically favorable amino acid substitutions in AB, EF, and CD loops; iii) the introduction of sequence modifications to the amino acid sequence, length, or both, of the AB, EF, and CD loop sequences, based on structural assumptions in view of wild-type sequences; and iv) the incorporation of cognate epitopes for commercially available antibodies to replace the amino acid sequence of the CD loop.

Example 1. Ig fold protein-derived epitope tags. Briefly, sequence modifications within the AB, CD, and EF loops were based on the substitution of AB, CD, or EF loop wild-type sequences with sequences derived from corresponding structural regions of other structurally-related Ig-fold proteins. This approach resulted in the identification of unique epitopes for incorporation into human CH3 domains. Sequence modifications of AB loop were generated in the context of the G1m1, as well as, the nG1m1 allotypes, (DEL vs EEM, respectively).

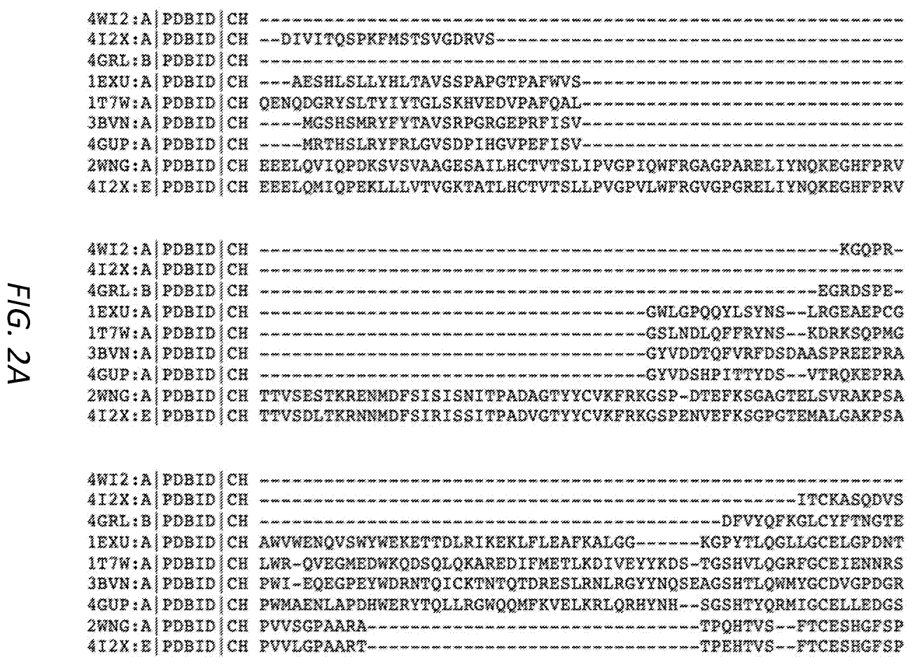

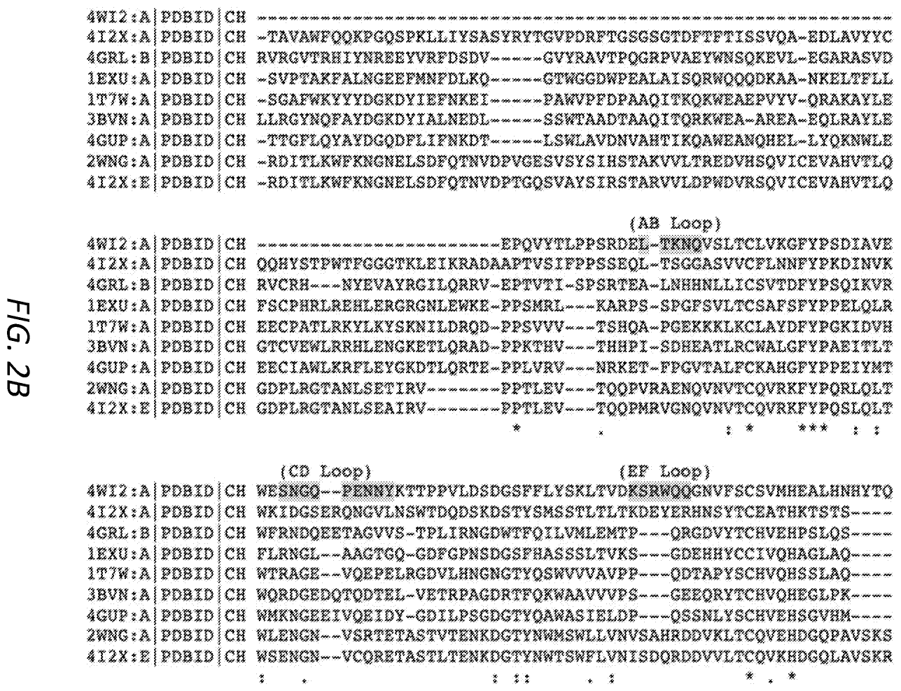

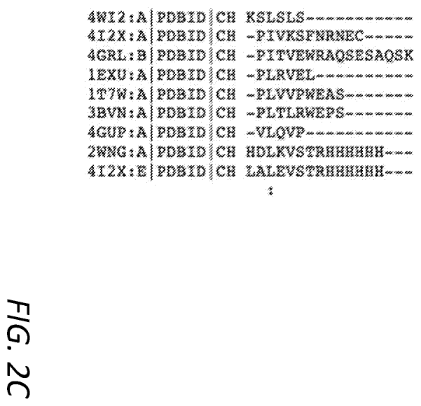

Candidate Ig-fold CH3 loop sequences corresponding to AB, EF, and CD loop sequences were identified by performing a multi-sequence alignment of the primary amino acid sequences of various Ig fold proteins against the sequence of the CH2 and CH3 domains of a human IgG1 Fc-region derived from the crystal structure associated with PDB 4W12, as summarized in FIGS. 2A-2C. The alignment included eight Ig-fold proteins whose crystal structures were available in the Research Collaboratory for Structural Bioinformatics Protein Data Bank (“PDB”), as of 11 Apr. 2018. See http://www.rcsb.ord. The PDB Ids of candidate Ig-fold proteins were: 2WNG; 412X:A; 412X:E; 4GRL; 1EXU; 1T7W; 3BVN; and 4GUP.

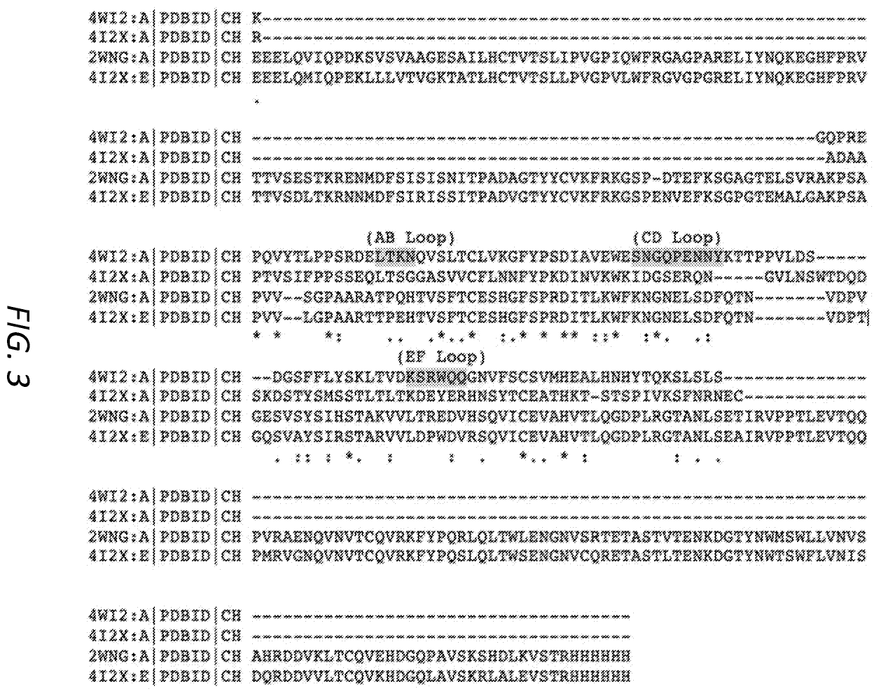

The alignment analysis showed general conservation of the spacing of the AB, CD, and EF loops within the Ig-fold proteins. Sequence motifs or “anchor residues” were present between each of the loops. Absolute sequence identity was low between proteins. Multisequence alignment of a subset of those proteins, specifically 412X:A, 412X:E, and 2WNG, identified a region of higher sequence identity with the CH3 domain of 4W12 (FIG. 3).

The alignment analysis considered several general classes of sequence position conservation, including spacing within regions of Ig folds, charge, isoelectric point (p1), and polarity. Although emphasis was placed on absolute identity of potential conserved anchor residues, such as Valine-Serine, C-terminal to the AB loop, and the Tryptophan at two residues N-terminal to the CD loop. The amino acid sequences of proteins found in two crystals, 2WNG and 412X, were more conserved with the IgG1 CH3 sequence at the AB, CD, and EF loops than the other six Ig-fold proteins. The crystal 2WNG corresponds with the regulatory membrane glycoprotein, signal regulatory protein alpha (SIRPα). The crystal 412X contains two Ig-fold proteins, 412X:A and 412X:E, corresponding to the light chain of an antibody Fab fragment and SIRP gamma (SIRPγ), respectively.

CH3 scaffolds based on the amino acid sequence derived from the PDB 4W12 IgG1 crystal were designed to incorporate the regions of SIRPα, SIRPγ, and the CL domain of 412X:A that correspond with the AB, CD, and EF loops of human CH3. Tables 1-3 summarize SIRPα, SIRPγ, and CL derived epitope tag amino acid sequences that were used to replace the wild-type (WT) sequences of the CD, AB, and EF loops, respectively. Amino acids in the wild type sequences with side chains pointing to the interior of the antibody structure were not substituted, however, because of their potential structural significance, as well as because those residues would not be not exposed and, as such, would not be accessible by the detection antibody.

TABLE 1

| | Loop AA | |
|---|---|---|
| | sequence | |
| | Bordered | |
| Modifi- | (amino/carboxy) | |
| cation | by WE/KTT | Description |
| CD-WT | SNGQPENNY | Wild-type |
| | (SEQ ID NO. 2) | CD loop |
| | | sequence |
| CD-2WNG | NGNELSDF | SIRPα |
| | (SEQ ID NO. 4) | derived |
| | | CD loop |
| | | sequence |
| CD-4I2X:A | IDGSERQNG | CL derived |
| | (SEQ ID NO. 6) | CD loop |
| | | sequence |
| CD-4I2X:E | NGNELSDF | SIRPγ |
| | (SEQ ID NO. 4) | derived |
| | | CD loop |
| | | sequence |

TABLE 2

| | Loop AA | |
|---|---|---|
| | sequence | |
| | Bordered | |
| | (amino/carboxy) | |
| | by RDE/QVS | |
| | in G1m1 | |
| | allotype or | |
| Modifi- | REE/QVS in | |
| cation | nG1m1 allotype | Description |
| AB-WT | LTKN | Wild-type |
| | (SEQ ID NO. 32) | AB loop |
| | | sequence |
| AB-2WNG | TPQH | SIRPα |
| | (SEQ ID NO. 43) | derived AB |
| | | loop |
| | | sequence |
| AB-4I2X:A | LTSG | CL-derived |
| | (SEQ ID NO. 45) | AB loop |
| | | sequence |
| AB-4I2X:E | TPEH | SIRPγ |
| | (SEQ ID NO. 47) | derived AB |
| | | loop |
| | | sequence |

TABLE 3

| | Loop AA | |
|---|---|---|
| | sequence* | |
| | Bordered | |
| Modifi- | (amino/carboxy) | |
| cation | by D/GNV | Description |
| EF-WT | KSrwQQ | Wild-type |
| | (SEQ ID NO. 59) | EF loop |
| | | sequence |
| EF-2WNG | LTrwDV | SIRPα |
| | (SEQ ID NO. 61) | derived |
| | | EF loop |
| | | sequence |
| EF-4I2X:A | KDrwER | CL-derived |
| | (SEQ ID NO. 63) | EF loop |
| | | sequence |
| EF-4I2X:E | LDrwDV | SIRPγ derived |
| | (SEQ ID NO. 65) | EF loop |
| | | sequence |
| . | | |

*Amino acids with Ab interior-pointing side chains are indicated in lower case.

Example 2. Sterically favorable amino acid substitutions in AB and EF loops. Using the molecular visualization system software, PyMOL, human IgG1 Fc regions derived from crystal structures of PDB 4WI2 or 4NOU were used to model the location of the loops and identify solvent exposed amino acid side chains within the AB and EF loops. Using the mutagenesis feature of PyMOL to model amino acid substitutions, it was possible to analyze the steric effects of various amino acid substitutions of surface-exposed residues within the AB and EF loops. Substitutions that did not result in steric clashes were considered for additional analysis. Tables 4 and 5 contain candidate AB and EF epitope amino acid sequences, respectively, developed by identifying sterically-favorable substitutions in the surface-exposed loops of the AB and EF loops.

TABLE 4

| | Loop AA sequence | |
|---|---|---|
| | Bordered (amino/ | |
| | carboxy) by | |
| | RDE/QVS in G1m1 | |
| | allotype or | |
| | REE/QVS in | |
| Modification | nG1m1 allotype | Description |
| AB-WT | LTKN | Wild-type AB |
| | (SEQ ID NO. 32) | loop sequence |
| AB-ISRQ | ISRQ | Modified AB |
| | (SEQ ID NO. 41) | loop sequence |

TABLE 5

| | Loop AA sequence* | |
|---|---|---|
| | Bordered (amino/ | |
| Modifi- | carboxy) by | |
| cation | D/GNV | Description |
| EF-WT | KSrwQQ | Wild-type EF |
| | (SEQ ID NO. 59) | loop sequence |
| EF-ND | NDrwQQ | Modified EF |
| | (SEQ ID NO. 67) | loop sequence |

*Amino acids with Ab interior-pointing side chains are indicated in lower case.

Example 3. CD loop sequence modifications. Using the molecular visualization system software, PyMOL, human IgG1 Fc regions derived from crystal structures of PDB 4W12 or 4N0U were used to model the location of the loops and identify solvent exposed amino acid side chains within the CD loop sequence. Using that approach, it was possible to analyze the steric effects of sequence modifications within the context of the CD loop. Amino acid substitutions were selected based on similarities of charge, isoelectric point (pI), and polarity at each amino acid position. Another general consideration for the design of CD epitopes was to avoid a hydrophobic patch on the outside of the antibody. Table 6 contains a listing of candidate CD epitope tag sequences selected by employing the foregoing strategy.

TABLE 6

| | Loop AA sequence | |
|---|---|---|
| Modifi- | Bordered (amino/ | |
| cation | carboxy) by WE/KTT | Description |
| CD-WT | SNGQPENNY | Wild-type CD |
| | (SEQ ID NO. 2) | loop sequence |
| CD-TLK | DNPVY | Modified CD |
| | (SEQ ID NO. 8) | loop sequence |
| CD-CD2HV | SNIAQPRNY | Modified CD |
| | (SEQ ID NO. 10) | loop sequence |
| CD-KRNE | SNGQPEKRNENNY | Modified CD |
| | (SEQ ID NO. 12) | loop sequence |
| CD-LANE | SNGQPELANENNY | Modified CD |
| | (SEQ ID NO. 14) | loop sequence |
| CD-DRR | SNGQPDRRY | Modified CD |
| | (SEQ ID NO. 16) | loop sequence |
| CD-DQQ | SNGQPDQQY | Modified CD |
| | (SEQ ID NO. 20) | loop sequence |

Example 4. Incorporation of cognate epitopes for commercially available antibodies to replace the amino acid sequence of the CD loop. Table 7 contains descriptions of CD loops that were modified by replacing the wild type amino acid sequence with known epitope tag sequences recognized by commercially available antibodies.

TABLE 7

| | Loop AA sequence | |
|---|---|---|
| Modifi- | Bordered (amino/ | |
| cation | carboxy) by WE/KTT | Description |
| CD-WT | SNGQPENNY | Wild-type CD |
| | (SEQ ID NO. 2) | loop sequence |
| CD-OPN | TWLNPDPSQ | known epitope- |
| | (SEQ ID NO. 22) | derived sequence |
| CD-Glu | YMPMENNY | known epitope- |
| | (SEQ ID NO. 24) | derived sequence |
| CD-MYC | QKLISEEDL | known epitope- |
| | (SEQ ID NO. 26) | derived sequence |
| CD-FLAG | DYKDDDD | known epitope- |
| | (SEQ ID NO. 28) | derived sequence |
| CD-HIS | SNGHHHHHHY | known epitope- |
| | (SEQ ID NO. 30) | derived sequence |

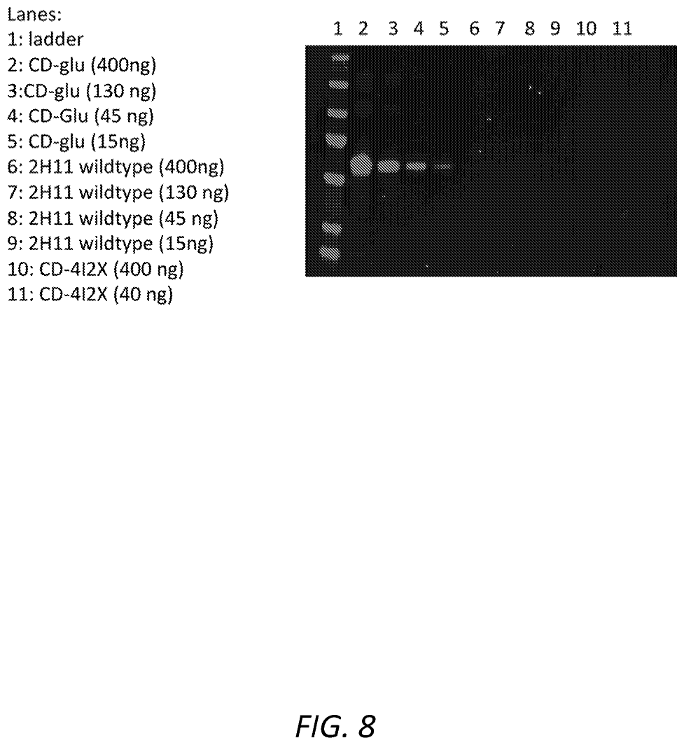

Incorporation of the CD-Glu epitope into the m1,17 backbone allows for detection of this antibody with commercially available anti-GLU antibodies (SigmaAldrich, Cat #AB3788). As depicted in FIG. 8, CD-GLU is selectively detected via Western blot as compared to either an antibody containing a wild-type m1,17 CD loop (CD-WT) or one comprising the CD-412X:E epitope. CD-GLU was detectable by Western blot over a range of, at least, 15-400 ng. No signal was detected for either CD-WT or CD-412X:E at levels as high as 400 ng.

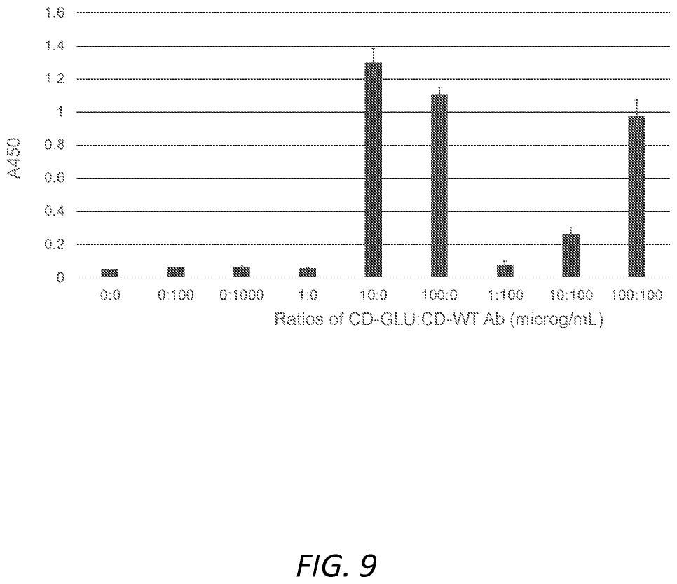

CD-GLU can be detected in the presence of CD-WT antibody. CD-GLU and CD-WT were mixed in phosphate buffered saline at ratios of 0:100, 0:1000, 1:0, 10:0, 100:0, 1:100, 10:100, 100:100 (μg/mL CD:-GLU: μg/mL CD-WT) and coated onto the surface of an ELISA plate in triplicate. Phosphate buffered saline without either CD-GLU or CD-WT (0:0) served as background control. Bound CD-GLU was detected with 1:1000 dilution of anti-CDGLUGLU polyclonal antisera (SigmaAldrich, Cat #AB3788) as primary and a 1:5000 dilution of mouse anti-rabbit-IgG-HRP conjugated secondary antibody (Southern Biotech, Cat #4090-05). Signal was developed with OPD substrate and absorbance was read at 450 nm. As depicted in FIG. 9, CD-WT was not detected above background levels when plated at concentrations as high as 1000 μg/mL. In contrast, CD-GLU was detectable at concentrations as low as 10 μg/mL when plated in the absence of CD-WT. CD-GLU, at concentrations of 10 and 100 μg/mL was also detected in the presence of 100 μg/mL CD-WT.

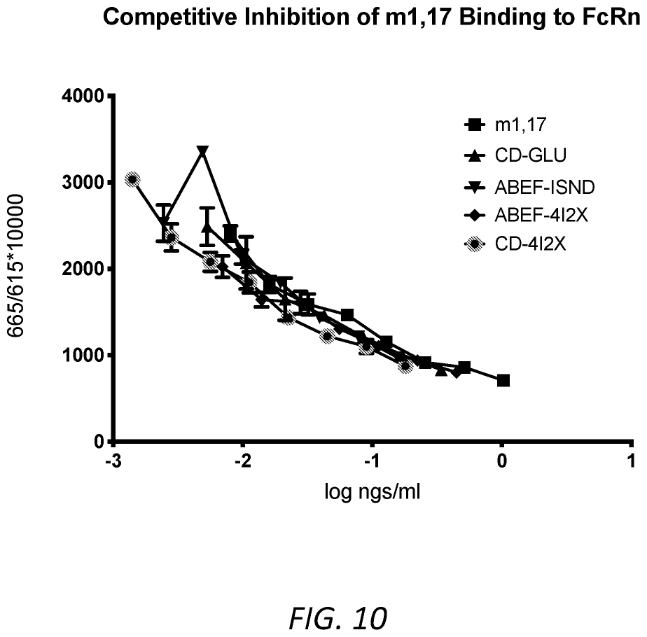

Example 5. Incorporation of epitopes does not dramatically alter binding to neonatal Fc receptor (FcRn). Binding affinity for FcRn correlates with the pharmacokinetics (PK) behavior, and thus serum half-life, of antibodies. To address whether the designed epitopes alter the ability of IgG to bind to FcRn, and thus have an impact on in vivo PK, a panel of antibodies containing the CD-WT, CD-GLU, CD-412X, ABEF-ISND, and ABEF-4I2X:E epitope tags were expressed, in the context of the same variable domains, and assessed for binding to FcRn using a competitive FRET-based assay (Cisbio). As demonstrated in FIG. 10, CD-WT is able to effectively compete binding of donor-labeled human IgG in a dose-dependent manner. Incorporation of any of the four epitopes tested did not dramatically alter the ability of antibody to compete with donor-labelled human IgG. These data suggest, as predicted by the design processes, that incorporation of epitopes into either the CD or ABEF loops, sites that are distinct from the known interation sites with FcRn, do not significantly alter the ability of the antibodies to bind to FcRn. Therefore, incorporation of epitopes into these loops is not predicted to alter PK of the antibodies.

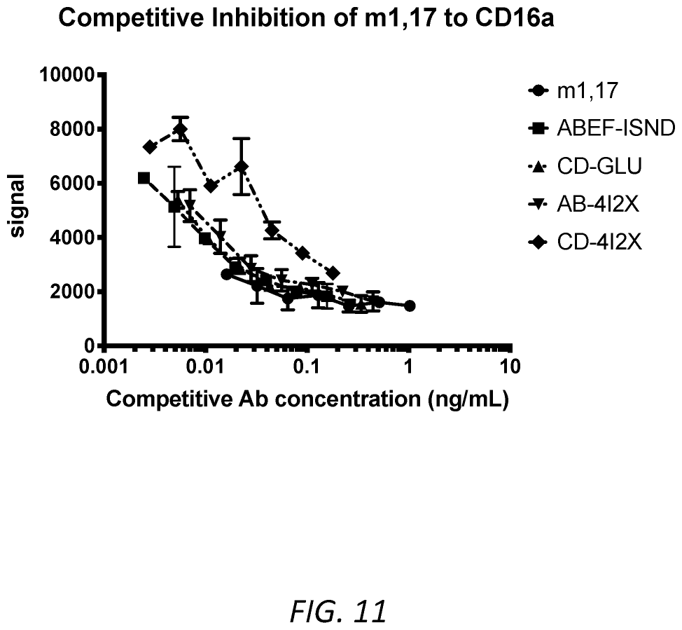

Example 6. Binding to FcγReceptors differentiates between epitopes. Different classes of immune effector cells express unique combinations of FcγRs on their cell surface. Engagement of those FcγRs by antibodies, through their Fc domains, modulates activity of the immune effector cells. For example, engagement of CD16a (FcγRIIIa) on the surface of natural killer (NK) cells, is important for inducing antibody-dependent cellular cytotoxicity (ADCC). Naturally occuring polymorphisms in CD16a, for example CD16aV158F and CD16aV176F, alter the binding affinity of the receptor for the Fc domains of IgG molecules. This in turn alters the ability of IgG to induce ADCC in vitro and has been correlated with clinical response to some antibody-based therapeutics. Using a competitive FRET assay (Cisbio), the ability of CD-WT Fc, in the context of the m1,17 allotype, as well as CD-GLU, CD-412X:E, ABEF-ISND, and ABEF-412X:E were assessed for binding to CD16a158V. As shown in FIG. 11, CD-GLU, ABEF-ISND, and ABEF-412X:E were able to compete with human IgG for binding to CD16a158V in a dose-dependent manner that mimiced that observed with the antibody containing a wild-type (m1,17 allotype) Fc domain. In contrast, the CD-412X:E antibody required approximately a 10-fold greater concentration to achieve the same level of competition, consistent with a decreased affinity for the CD16a158V receptor.

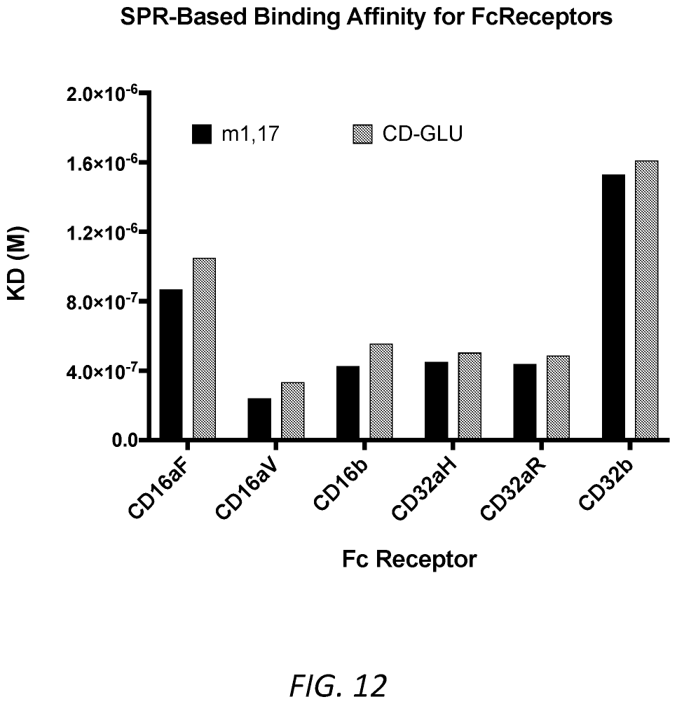

Additional members of the FcγR family exist on various subsets of immune effector cells. Among those are CD16b (FcγRIIIb), CD32a (FcγRIIa), CD32b (FcγRIIb), and CD64 (FcγRI). CD32 and CD16b are low affinity IgG receptors, CD16a binds with intermediate affinity, and CD64 binds with high affinity to monomeric IgG. As with CD16a, polymorphisms, such as CD32aH131R, exist in other FcγRs that alter binding affinity. Wild-type (CD-WT) and CD-GLU were further characterized using surface plasmon resonance, on a BIAcore 8K, to measure, and generate a direct comparison of, affinity for CD16a176V, CD16a176F, CD16b, CD32a167H, CD32a167R, and CD32b. Approximately 150 RU CD-WT and CD-GLU were captured on an anti-Fab immobilized Series S CM5 sensor chip, with the goal of generating an Rmax of 50-100 RU upon binding to FcγRs. Affinity for each of the FcγRs were measured using a minimum of 10 replicates and assessed using an affinity model specific for the individual FcγRs. Single-cycle kinetics with a bivalent analyte model was used for CD64 and CD16 family receptors and steady state binding was used to assess binding to the CD32 family of receptors. Results of those studies are depicted in Tables 8 and 9, as well as FIGS. 12 & 13. Overall, data reflects similar binding by CD-WT and CD-GLU, with a possible trend toward CD-GLU having a modest decrease, ranging from 1.1-1.4 fold for all families of FcγRs. In the case of FcγR1 binding, data point to a small, but consistent, increase in off-rate for CD-GLU relative to CD-WT being responsible for the difference. Binding to FcγR1 is known to be impacted by glycosylation state of the Fc domain. Slight differences in glycosylation of the CD-GLU, relative to CD-WT, may be responsible for the observed difference.

TABLE 8
Bivalent Analyte Model

| Sample | Fc Receptor | Chi² | ka1 (1/Ms) | kd1 (1/s) | KD1 (M) | Rmax (RU) |
|---|---|---|---|---|---|---|
| CD-WT | CD16aF | 2.09E+00 | 2.13E+05 | 1.85E−01 | 8.69E−07 | 81.1 |
| | CD16aV | 4.81E+00 | 7.51E+05 | 1.81E−01 | 2.41E−07 | 88.6 |
| | CD16b | 5.83E+00 | 3.89E+05 | 1.66E−01 | 4.26E−07 | 89.6 |
| CD-Glu | CD16aF | 1.08E+00 | 1.81E+05 | 1.89E−01 | 1.05E−06 | 70.8 |
| | CD16aV | 2.46E+00 | 4.38E+05 | 1.46E−01 | 3.34E−07 | 79.2 |
| | CD16b | 2.75E+00 | 3.02E+05 | 1.68E−01 | 5.56E−07 | 80.1 |

TABLE 9
Study State Affinity Fits

| Sample | Fc Receptor | Chi² | KD (M) | Rmax (RU) | offset (RU) |
|---|---|---|---|---|---|
| CD-WT | CD32aH | 9.41E−01 | 4.50E−07 | 70.9 | 4.4 |
| | CD32aR | 9.37E−01 | 4.39E−07 | 64.2 | 2.1 |
| | CD32b | 6.12E−01 | 1.53E−06 | 54.2 | 2.6 |
| CD-Glu | CD32aH | 4.41E−01 | 5.05E−07 | 63.3 | 3.3 |
| | CD32aR | 1.01E−01 | 4.88E−07 | 58.0 | 0.9 |
| | CD32b | 9.69E−02 | 1.61E−06 | 47.2 | 1.7 |

Example 7. Immunogenicity prediction of CD-GLU. Modification of antibody sequences, even the use of different naturally occurring allotype backbones, has been associated with altered rates of immunogenicity. To evaluate the potential immunogenicity risk of incorporating the CD-GLU epitope tag into the m1,17 backbone, the amino acid sequence was computationally analyzed for the introduction of MHC Class I and Class II binding peptides, using the NetMHCcons and NetMHCIIpan (version 3.2) servers, respectively. The analyses provide prediction values, given in nM binding affinity and as % Rank compared to a set of 200,000 random natural peptides for strong and weak binding peptides. NetMHCcons evaluated 321, nine amino acid peptides that shifted in register by one amino acid across the amino acid sequence of the m1,17 allotype Fc, with the incorporated CD-GLU epitope tag (SEQ ID NO: 73), which spans the CH1, CH2, and CH3 domains of a human IgG1. NetMHCIIpan evaluated 315, 15 amino acid peptides across the same CD-GLU containing heavy chain sequence. Strong MHC I binding peptides are those with 50 nM affinity and within the top 0.5% relative to control peptides. Weak MHC I binding peptides are those with 500 nM affinity and within the top 2%. Strong and weak MHC II binding peptides are those within the top 2% and 10%, respectively, relative to the naturally occurring control set. The algorithms predicted three strong and three weak MHC I binding peptides, as well as zero strong and 11 weak MHC II binding peptides. Examples of strong and weak MHC I binding peptides are peptides 133 and 116, respectively. Likewise, a peptide predicted to bind weakly to MHCII is peptide 178. All of the predicted peptides are located in naturally occurring areas of the wild-type Fc domain. Insertion of the CD-GLU epitope introduce neither an MHC I nor an MHC II binding peptide. Results for overlapping peptides, containing any portion of the CD-GLU epitope, are listed in Table 10 and 11.

TABLE 10
MHCI binding of peptides across
CD-GLU insert in CH3 domain

| Seq | | | |
|---|---|---|---|
| Binding | Peptide | Affinity | % |
| Level | Sequence | (nM) | Rank |
| 133 | LMISRTPEV | 4.6 | 0.1 |
| 116 | LLGGPSVFL | 101.5 | 3 |
| 257 | SDIAVEWEY | 29266.51 | 50 |
| 258 | DIAVEWEYM | 25152.75 | 50 |
| 259 | IAVEWEYMP | 21617.22 | 32 |
| 260 | AVEWEYMPM | 11922.2 | 32 |
| 261 | VEWEYMPME | 26694.99 | 50 |
| 262 | EWEYMPMEN | 36534.22 | 50 |
| 263 | WEYMPMENN | 34798.02 | 50 |
| 264 | EYMPMENNY | 34237.82 | 50 |
| 265 | YMPMENNYK | 17505.31 | 32 |
| 266 | MPMENNYKT | 17505.31 | 32 |
| 267 | PMENNYKTT | 35367.39 | 50 |
| 268 | MENNYKTTP | 34798.02 | 50 |
| 269 | ENNYKTTPP | 35559.24 | 50 |
| 270 | NNYKTTPPV | 10191.11 | 15 |
| 271 | NYKTTPPVL | 32965.5 | 50 |
| 272 | YKTTPPVLD | 36141.06 | 50 |

TABLE 11
MHCII binding of peptides across
CD-GLU insert in CH3 domain

| Seq | | | | |
|---|---|---|---|---|
| Bind- | | | | |
| ing | Peptide | | Affinity | % |
| Level | Sequence | Core | (nM) | Rank |
| 178 | QYNSTYRVVSVLTVL | YRVVSVLTV | 34.72 | 8 |
| 252 | VKGFYPSDIAVEWEY | FYPSDIAVE | 311.06 | 49 |
| 253 | KGFYPSDIAVEWEYM | FYPSDIAVE | 417.88 | 55 |
| 254 | GFYPSDIAVEWEYMP | FYPSDIAVE | 755.09 | 70 |
| 255 | FYPSDIAVEWEYMPM | DIAVEWEYM | 1266.25 | 80 |
| 256 | YPSDIAVEWEYMPME | DIAVEWEYM | 2186.2 | 90 |
| 257 | PSDIAVEWEYMPMEN | DIAVEWEYM | 2298.01 | 90 |
| 258 | SDIAVEWEYMPMENN | WEYMPMENN | 1377.24 | 80 |
| 259 | DIAVEWEYMPMENNY | WEYMPMENN | 767.61 | 70 |
| 260 | IAVEWEYMPMENNYK | YMPMENNYK | 224.09 | 41 |
| 261 | AVEWEYMPMENNYKT | YMPMENNYK | 125.81 | 29 |
| 262 | VEWEYMPMENNYKTT | YMPMENNYK | 102.83 | 25 |
| 263 | EWEYMPMENNYKTTP | YMPMENNYK | 120.35 | 28 |
| 264 | WEYMPMENNYKTTPP | YMPMENNYK | 133.83 | 30 |
| 265 | EYMPMENNYKTTPPV | YMPMENNYK | 239.99 | 43 |
| 266 | YMPMENNYKTTPPVL | YMPMENNYK | 341.44 | 55 |
| 267 | MPMENNYKTTPPVLD | YKTTPPVLD | 437.76 | 60 |
| 268 | PMENNYKTTPPVLDS | YKTTPPVLD | 239.5 | 43 |
| 269 | MENNYKTTPPVLDSD | YKTTPPVLD | 294.67 | 48 |
| 270 | ENNYKTTPPVLDSDG | YKTTPPVLD | 349.21 | 55 |
| 271 | NNYKTTPPVLDSDGS | YKTTPPVLD | 388.13 | 55 |
| 272 | NYKTTPPVLDSDGSF | YKTTPPVLD | 705.32 | 70 |
| 273 | YKTTPPVLDSDGSFF | YKTTPPVLD | 2961.71 | 90 |

Example 8. CD-GLU containing Fc domain retains thermal stability. Differential scanning fluorimetry was performed on two antibodies that differ only in the presence or absence of CD-GLU epitope. Both antibodies were formulated in 100 mM HEPES, 100 mM NaCl, 50 mM NaOAc, pH6.0 at concentrations of 1.55 mg/mL (CD_GLU) or 1.97 mg/mL (CD-WT). Thermal stability was analyzed by differential scanning fluorimetry under a temperature ramp of 1° C./min from 25° C. to 95° C. The first derivative plot of change in fluorescence over change in temperature (dFluor/dTemperature, nm/° C.) was plotted to define melting temperatures (Tms). As defined in Table 12, CD-WT had three and CD-GLU had two melting point transitions, consistent with known melting of IgG molecules. The 75.2° C. melting point, likely to correspond to the Fc domain of CD-GLU, was approximately six degrees lower than that measured for CD-WT. Although lower than that of CD-WT, the observed temperature is consistent with that seen in other clinically relevant antibodies (Andersen et al),

TABLE 12
Impact of CD-GLU on thermal stability of antibody

| Antibody | Tm1 (° C.) | Tm2 (° C.) | Tm3 (° C.) |
|---|---|---|---|
| CD-GLU | 66.1 | 75.2 | |
| CD-WT | 68.7 | 81.5 | 84.6 |

### REFERENCES

Andersen C B, Manno M, Rischel C et al (2010) Aggregation of a multidomain protein: a coagulation mechanism governs aggregation of a model IgG1 antibody under weak thermal stress. Protein Sci. 19:279-290.
Jefferis and Lefranc (2009) Human immunoglobulin allotypes: Possible implications for immunogenicity. mAbs. 1(4):332-338.

What is claimed is:

1. A CH3 scaffold comprising at least one antibody epitope amino acid sequence, wherein at least one antibody epitope amino acid sequence comprises at least one modification of the wild-type amino acid sequence of the CH3 domain derived from an immunoglobulin Fc region.

2. The CH3 scaffold according to claim 1, wherein at least one modification of the wild-type sequence occurs within the AB, EF, or CD loops of the CH3 scaffold.

3. The CH3 scaffold according to claim 2, wherein the at least one modification is an amino acid substitution, deletion or insertion.

4. The CH3 scaffold according to claim 3, wherein the at least one antibody epitope amino acid sequence is located within the AB loop.

5. The CH3 scaffold according to claim 4, wherein the antibody epitope amino acid sequence comprises a sequence derived from SIRPα or SIRPγ.

6. The CH3 scaffold according to claim 4, wherein the antibody epitope amino acid sequence comprises a sequence derived from a constant light chain of an antibody.

7. The CH3 scaffold according to claim 4, wherein the antibody epitope amino acid sequence comprises a sequence selected from the group consisting of SEQ ID Nos. 33-57.

8. The CH3 scaffold according to any one of claims 4-7, wherein the EF and CD loops comprise only wild-type amino acid sequences.

9. The CH3 scaffold according to claim 3, wherein the at least one antibody epitope amino acid sequence is located within the EF loop.

10. The CH3 scaffold according to claim 9, wherein the antibody epitope amino acid sequence comprises a sequence derived from SIRPα or SIRPγ.

11. The CH3 scaffold according to claim 9, wherein the antibody epitope amino acid sequence comprises a sequence derived from a constant light chain of an antibody.

12. The CH3 scaffold according to claim 9, wherein the single antibody epitope amino acid sequence comprises a sequence selected from the group consisting of SEQ ID Nos. 60-67.

13. The CH3 scaffold according to any one of claims 9-12, wherein the AB and CD loops comprise only the wild-type amino acid sequences.

14. The CH3 scaffold according to claim 4, wherein the antibody epitope amino acid sequence is located within the CD loop.

15. The CH3 scaffold according to claim 14, wherein the single antibody epitope amino acid sequence comprises a sequence derived from SIRPα or SIRPγ.

16. The CH3 scaffold according to claim 14, wherein the antibody epitope amino acid sequence comprises a sequence derived from a constant light chain of an antibody.

17. The CH3 scaffold according to claim 14, wherein the antibody epitope amino acid sequence comprises a sequence selected from the group consisting of SEQ ID Nos. 3-30.

18. The CH3 scaffold according to any one of claims 14-17, wherein the AB and EF loops comprise only the wild-type amino acid sequences of the immunoglobulin heavy chain.

19. The CH3 scaffold according to any one of claims 1-18, wherein the CH3 scaffold is derived from a human immunoglobulin Fc region.

20. The CH3 scaffold according to claim 19, wherein the human antibody is an IgG1, IgG2, IgG3, or IgG4.

21. The CH3 scaffold according to claim 20, wherein the human antibody is an IgG1.

22. The CH3 scaffold according to claim 21, wherein the IgG1 is a G1m1 or nG1m1 allotype.

23. A human antibody or portion thereof comprising a CH3 scaffold according to any one of the claims 1-22.

24. The human antibody or portion thereof according to claim 23, wherein the antibody is an IgG1, IgG2, IgG3, or IgG4.

25. The antibody or portion thereof according to claim 23 or 24, wherein the antibody or portion thereof wherein, with the exception of one or more its CDRs and one or more of its antibody epitope amino acid sequences, is humanized.

25. An engineered human Fc region or portion thereof comprising a CH3 scaffold according to any one of the claims 1-22.

26. The Fc region or portion thereof according to claim 25, wherein Fc region is derived from a human antibody.

27. The Fc region or portion thereof according to claim 26, wherein the human antibody is IgG1, IgG2, IgG3, or IgG4.

28. The Fc region or portion thereof according to claim 27, wherein the human antibody is IgG1.

29. The Fc region or portion thereof according to claim 27, wherein the IgG1 is a G1m1 or nG1m1 allotype.

30. An antibody or portion thereof, wherein the antibody is specific for an antibody epitope sequence according to any one of claims 1-29.

31. A method for engineering a CH3 scaffold having AB, EF, and CD structural loop regions, wherein at least one of the structural loop regions comprises an antibody epitope amino acid sequence, wherein the antibody epitope amino acid sequence comprises at least one modification of the wild-type sequence within the structural loop region, comprising the following steps:
(i) providing a nucleic acid molecule encoding a CH3 scaffold having AB, EF, and CD structural loop regions;
(ii) modifying the nucleic acid sequence encoding at least one of the AB, EF, and CD structural loop regions;
(iii) transferring the modified nucleic acid molecule into an expression system;
(iv) expressing the modified CH3 scaffold encoded by the nucleic acid sequence modified according to step (ii).

32. The method for engineering a CH3 scaffold according to claim 31, wherein the amino acid sequence modification is an amino acid substitution, deletion or insertion.

33. The method for engineering a CH3 scaffold according to claim 31, wherein the antibody epitope amino acid sequence comprises a sequence derived from SIRPα or SIRPγ.

34. The method for engineering a CH3 scaffold according to claim 31, wherein the antibody epitope amino acid sequence comprises a sequence derived from a constant light chain of an antibody.

35. The method for engineering a CH3 scaffold according to claim 31, wherein the antibody epitope amino acid sequence is located in the AB loop, and comprises a sequence selected from the group consisting of SEQ ID Nos. 33-57.

36. The method for engineering a CH3 scaffold according to claim 31, wherein the antibody epitope amino acid sequence is located in the EF loop, and comprises a sequence selected from the group consisting of SEQ ID Nos. 60-67.

37. The method for engineering a CH3 scaffold according to claim 31, wherein the antibody epitope amino acid sequence is located in the CD loop, and comprises a sequence selected from the group consisting of SEQ ID Nos. 3-30.

38. The method for engineering a CH3 scaffold according to claim 31, wherein the CH3 scaffold is derived from a human IgG antibody.

39. The method for engineering a CH3 scaffold according to claim 31, wherein the human IgG antibody is an IgG1, IgG2, IgG3, or IgG4.

40. The method for engineering a CH3 scaffold according to claim 39, wherein the CH3 scaffold is derived from an IgG1.

41. The method for engineering a CH3 scaffold according to claim 40, wherein the IgG1 expresses the G1m1 or nG1m1 allotype.

42. The method engineering a CH3 scaffold according to any one of claims 31-41, wherein the expression system comprises the nucleic acid sequence of an IgG Fc region selected from IgG1, IgG2, IgG3, or IgG4, and wherein the nucleic acid molecule encoding a CH3 scaffold is positioned in the expression system to fully or partially replace the nucleotide sequence of a wild type CH3 domain.
